(19) United States
(12) Patent Application Publication
(10) Pub. No.: US 2019/0024030 A1
(43) Pub. Date: Jan. 24, 2019

(54) METHOD AND SYSTEM TO EVALUATE EMBRYOS

(71) Applicant: EMBRYOTICS LLC, Granbury, TX (US)

(72) Inventors: Justin WELLS, Dripping Springs, TX (US); Cara WESSELS, Dripping Springs, TX (US); Robert RANGEL, Granbury, TX (US)

(73) Assignee: EMBRYOTICS LLC, Granbury, TX (US)

(21) Appl. No.: 16/139,179

(22) Filed: Sep. 24, 2018

Related U.S. Application Data
(63) Continuation-in-part of application No. 15/485,683, filed on Apr. 12, 2017, now abandoned.
(60) Provisional application No. 62/562,639, filed on Sep. 25, 2017.

(51) Int. Cl. C12M 3/00; C12M 1/36; C12M 1/34; G01N 33/483; G01N 33/50; G01N 15/14; G06T 7/00

(52) U.S. Cl. CPC C12M 21/06; C12M 41/48; C12M 41/46; G01N 2015/1075; G01N 33/5005; G01N 15/1475; G06T 7/0012; G01N 33/4833

(57) ABSTRACT

A system to evaluate an embryo is provided. The system includes a measurement chamber having a bottom end. The measurement chamber is configured to receive an embryo such that the embryo descends towards the bottom end, and a culture medium is disposed within the measurement chamber. At least one sensor is configured to assess the embryo descending towards the bottom end and output a data signal representative of at least one characteristic of the embryo descending through at least a portion of the measurement chamber. A processor receives the data signal from the at least one sensor and determines one or more embryo properties based on the at least one characteristic.

### CROSS-REFERENCE TO RELATED APPLICATIONS

This application claims priority to U.S. Provisional Patent Application No. 62/562,639, filed in the U.S. Patent and Trademark Office on Sep. 25, 2017, and U.S. patent application Ser. No. 15/485,683, filed in the U.S. Patent and Trademark Office on Apr. 12, 2017, each of which is incorporated herein by reference in its entirety for all purposes.

### FIELD

The present disclosure relates generally to animal breeding. In particular, the present disclosure relates to methods and systems to determine a viable and/or genetically desirable offspring.

### BACKGROUND

During animal breeding, controlled interaction between a male and female animal may be attempted to achieve desirable traits in the offspring. Often times, breeding is commenced without the ability to select desirable traits other than through selecting the animals. Additionally, some of the embryos can be damaged during the handling and freezing phases and become unviable or undesirable.

### BRIEF DESCRIPTION OF THE DRAWINGS

Implementations of the present technology will now be described, by way of example only, with reference to the attached figures, wherein:

FIG. 1 is a flowchart of a fertilization method;

FIG. 2A is a diagrammatic view of an exemplary embryo selection system;

FIG. 2B is a schematic diagram of a processing system which may be employed as shown in FIG. 2A;

FIG. 3 is a diagrammatic view of an exemplary storage platform;

FIGS. 4A and 4B are diagrammatic views of an exemplary door system;

FIG. 5 is a top view of another exemplary storage platform system; and

FIG. 6 is a flowchart of a method to determine embryo properties.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented. The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. For example, “substantially cylindrical” means that the object resembles a cylinder, but can have one or more deviations from a true cylinder. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

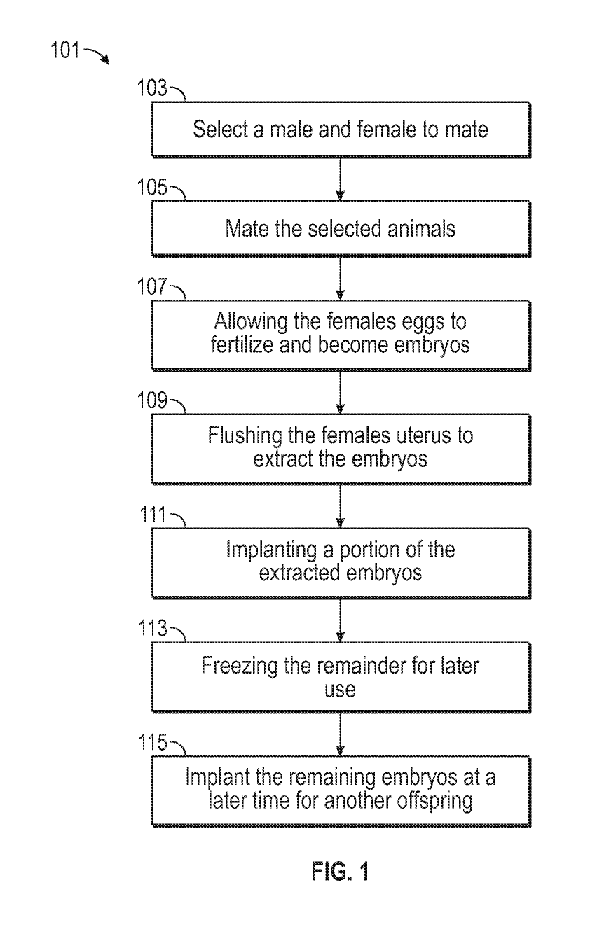

FIG. 1 is a flow chart depicting an exemplary fertilization method 101, which begins at block 103. At block 103, a male and a female are selected to mate. At block 105, the selected animals mate, allowing, at block 107, the female's eggs to fertilize and become embryos. In other examples, the selected animals do not mate, and the oocytes or eggs are fertilized by artificial insemination when preparing embryos to transfer. At block 109, the female's uterus is flushed to extract the embryos. At block 111, a portion of the extracted embryos may be implanted into the recipient female. At block 113, the remainder of the extracted embryos may be frozen for later use. At block 115, the remaining embryos may be implanted at a later time for another offspring. Throughout the method 101, embryos may have undesirable traits and/or be unviable.

Disclosed herein is a system and method using such a system which enables quantifiable measurements of embryos that facilitate selecting desirable offspring. In addition, damaged, unviable, or undesirable embryos can be removed from those to be implanted in the female. The system includes a measurement chamber at least partially filled with a culture medium. One or more embryos are inserted into the measurement chamber and, in the medium, descend towards the bottom of the measurement chamber. In at least one example, the culture medium is statically contained within the measurement chamber. For example, the culture medium is not under any force such as a propulsion system or a circulator and is not flowing. As such, the culture medium is static and the embryos pass through the static culture medium. One or more sensors assess the embryo descending through the measurement chamber data and output a data signal representative of at least one characteristic of the embryo descending through the portion of the measurement chamber. For example, the characteristics can include the descent rate, or descent distance versus time, of the embryos through the measurement chamber. In some examples, the characteristics may include weight, membrane integrity, biochemical properties, density, and/or specific gravity of the embryos. In at least one example, the embryos can descend through the measurement chamber under the force of gravity.

While embryos are discussed throughout the disclosure, the system can also be utilized for other cells such as haploid cells, diploid cells, mammalian cells, reptilian cells, amphibian cells, eukaryote cells, or somatic cells such as blood cells, skin cells, and liver cells.

The sensors can include a camera which is configured to locate and track the embryos as the embryos descend towards the bottom of the measurement chamber. The system includes a processor communicatively coupled with the sensors which receives the data signal from the sensors and determines one or more properties of the embryos based on the characteristics. For example, viable embryos or cells descend at an average predetermined rate while unviable embryos or cells descend at a rate faster or slower than the viable embryos or cells and outside of one standard deviation from the average rate. Also for example, X chromosomes are larger and heavier than Y chromosomes, so female embryos are larger and heavier than male embryos and descend at a different rate, for example a faster rate, than male embryos. As such, the processor can determine properties such as the viability and/or the sex of the embryo based on the descent rate. In at least one example, the system can be utilized to determine further properties such as embryo development potential, embryo biochemical composition, oocyte competency, embryo survival of cryopreservation, aneuploidy, or trisomy. As such, one can evaluate whether embryos have desired properties and select the desired embryos to be implanted in the female. As such, the selection of desirable offspring is facilitated by the system.

Additionally, in at least one example, the system can be utilized to promote growth and/or viability of the embryos. While embryos are commonly cultured under static conditions, microfluidic culture systems can provide a more optimal culture system to improve embryo development and produce healthier offspring. Kinetic movement can increase blastomere formation as well as blastocyst formation.

Mechanics may play a role in embryonic development, and applied mechanical forces in vitro may mimic the oviduct's physical stimulation as it peristaltically pumps the embryo in to the uterus. As such, by disposing embryos into a measurement chamber as discussed in the present disclosure, the kinetic movement of the embryos as the embryos descend through a culture medium under the force of gravity can mimic the oviduct's physical stimulation in a cost-effective manner. The embryos move through the culture medium in the measurement chamber instead of culture medium being pumped around a static embryo which may enhance embryo development.

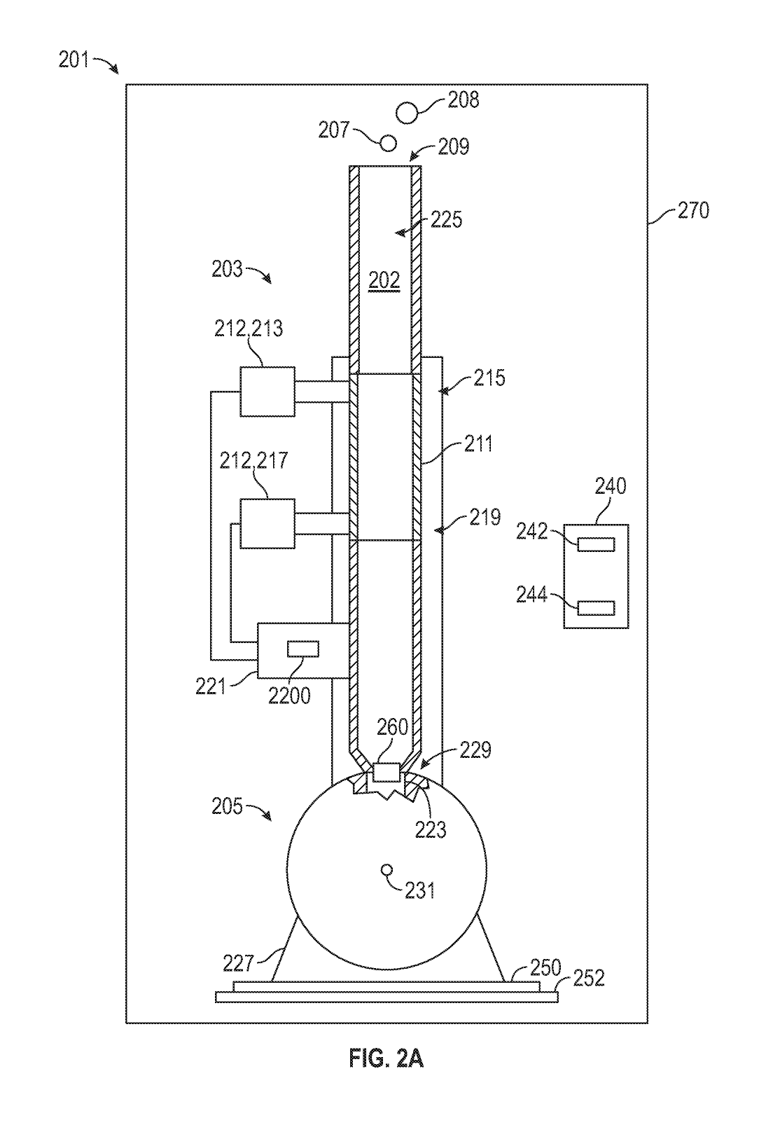

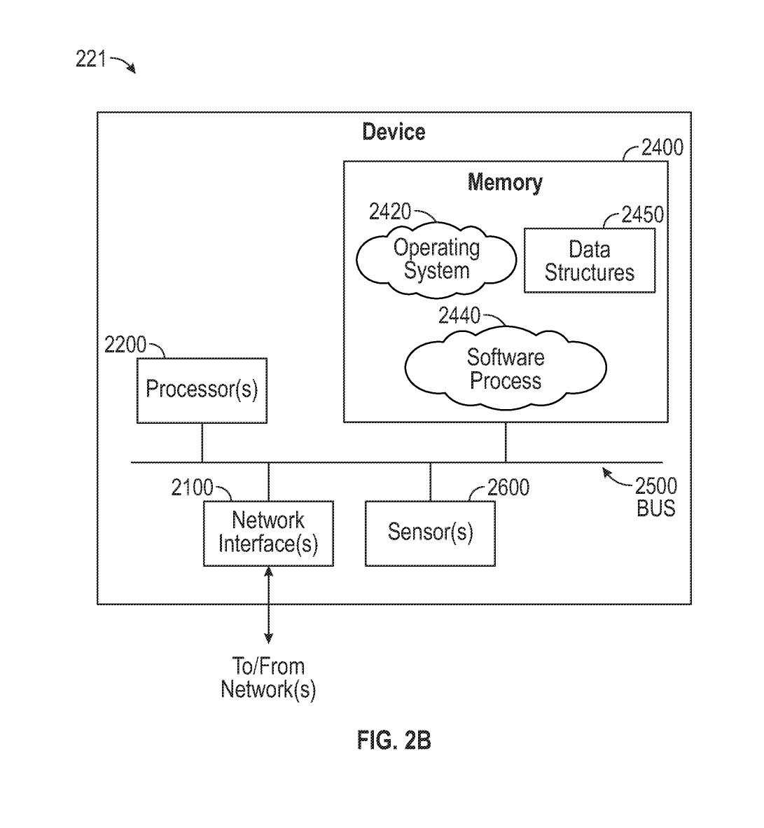

FIG. 2A illustrates a front view of an exemplary embryo selection system 201. System 201 includes a measurement chamber 203 in fluid communication with a storage platform 205 that enables embryos 207 to be tested and/or stored for selection. The measurement chamber 203, as illustrated in FIG. 2A, is a vertical column. The illustrated measurement chamber 203 is also substantially straight. In other examples, the measurement chamber 203 can be curved, have varying widths at different portions, or any other suitable shape. The measurement chamber 203 is filled with a culture medium 225 of known density and amiable to the survival of the embryos 207. The culture medium 225 can include water, protein, and energy to maintain the survival and/or growth of the embryos 207. For example, the culture medium 225 can include varying concentrations of glucose, lactate, pyruvate (energy sources), amino acids (protein), calcium and magnesium (metabolism and cellular functions). Antibiotics can be added to the culture medium 225 to prevent contamination.

The measurement chamber 203 includes a first end 209 and a second end 229. The first end 209 can be an opening in communication with the annulus 202 of the measurement chamber 203. The first end 209 is sized such that one or more embryos 207 can be inserted into the measurement chamber 203. For example, one embryo 207 may be inserted into the measurement chamber 203 at a time. In other examples, a plurality of embryos 207 may be inserted into the measurement chamber 203 at the same time. In at least one example, the first end 209 can be at the top end of the measurement chamber 203. In other examples, the first end 209 can be at any position of the measurement chamber 203 so long as one or more embryos 207 can be inserted into the measurement chamber 203 through the first open end 209. The second end 229, as illustrated in FIG. 2A, is at the bottom end of the measurement chamber 203. Similar to the first end 209, the second end 229 can be an opening in communication with the annulus 202 of the measurement chamber 203. The second end 229, as an opening, can be sized such that one or more embryos 207 can be exit from the measurement chamber 203.

In at least one example, the measurement chamber 203 can include at least one transparent portion 211 composed of a transparent material, such that the annulus 202 of the measurement chamber 203 can be visible through at least one side of the measurement chamber 203. In some examples, the transparent portion 211 can be a partial side of the measurement chamber 203. In other examples, the transparent portion 211 can traverse all sides of the measurement chamber 203. In some examples, the transparent portion 211 can have a height that is a portion of or an entirety of the height of the measurement chamber 203. In yet other examples, the measurement chamber 203 may not include a transparent portion 211.

In at least one example, a cryoprotectant 208 can be added into the measurement chamber 203. The cryoprotectant 208 can be layered onto the embryos 207 to protect the one or more embryos 208 during freezing and/or storage.

One or more sensors 212 are in communication with the measurement chamber 203. In some examples, the sensors 212 can be coupled with the measurement chamber 203. In some examples, the sensors 212 can be adjacent to but not directly in contact with the measurement chamber 203. The sensors 212 can be communicatively coupled with a processor 2200. In some examples, the processor 2200 may be provided within the system 201. In some examples, the processor 2200 may be remote in relation to the system 201. The sensors 212 are configured to assess the embryos 207 descending through at least a portion of the measurement chamber 203 and to output a data signal representative of at least one characteristic of the embryo 207 descending through the portion of the measurement chamber 203 to the processor 2200.

As illustrated in FIG. 2A, the system 201 includes one measurement chamber 203. In other examples, the system 201 can include a plurality of measurement chambers 203 so, for example, multiple tests can be conducted simultaneously.

In at least one example, the measurement chamber 203 can have a substantially circular cross-sectional shape. In other examples, the measurement chamber 203 can have a substantially rectangular or square cross-sectional shape. In yet other examples, the measurement chamber 203 can be any other suitable shape such as triangular, ovoid, or polygonal so long as one or more embryos 207 can pass through the annulus 202 of the measurement chamber 203 without interference.

As illustrated in FIG. 2A, the system 201 includes two sensors 212. In other examples, only one sensor 212 or more than two sensors 212 may be implemented. A first sensor 213 is rigidly attached proximate to the top end 215 of the transparent portion 211. A second sensor 217 is rigidly attached proximate to the bottom end 219 of the transparent portion 211. For example, the sensors 213, 217 can be lasers or any other suitable sensor to measure disruptions of the signal in the line of the sensors 213, 217. The sensors 213, 217 are in digital communication with the processor 2200 which monitors the sensors 213, 217 for disruptions and records the time between the interruptions. As such, by knowing the distance between the sensors 213, 217 and the time between the interruptions, the processor 2200 is able to calculate the descent rate of the embryo 207. For example, the distance measured by sensors 212 can be 1 centimeter.

An exemplary sensor 212 is a laser and exemplary materials for use in the transparent portion 211 are fiber optic wire, glass, or polystyrene; however other sensors and/or materials could be used. In at least one example, the measurement chamber 203 does not include a transparent portion 211, and the sensor(s) 212 can measure the descent rate of the embryos 207 without direct visibility from outside the measurement chamber 203. In some examples, the sensors 212 can be disposed within the measurement chamber 203, for example without the annulus 202 and/or disposed within the walls of the measurement chamber 203.

The processor 2200 can be any device or system capable of receiving information from sensors 212 for monitoring. For example, an exemplary system can include an amplifier configured to transmit information to a logic board for further calculations and information display. An exemplary processing system 221 which includes processor 2200 is discussed below in FIG. 2B.

In at least one example, the sensor(s) 212 can be configured to locate the embryos 207 and track the descent and/or movement of the embryos 207. For example, the sensor(s) 212 can include a camera which, coupled with the processor 2200, is configured to visually assess the embryos 207, for example by locating the embryos 207 and tracking the descent of the embryos 207 without the assistance of an operator. In other examples, the sensor(s) 212 can include radar detection devices which can assess the embryos 207 descending through the measurement chamber 203 without requiring a transparent portion 211 for visible access of the annulus 202 of the measurement chamber 203. Additionally, the system 201 can include one or more lights to provide illumination of the annulus 202 of the measurement chamber 203 to provide better visibility.

As illustrated in FIG. 2A, the system 201 includes a storage platform 205 including a plurality of receptacles 223 in communication with the second end 229 of the measurement chamber 203 for receiving and storing embryos 207. In some examples, the second end 229 of the measurement chamber 203 is closed such that the embryos 207 remain within the measurement chamber 203 for storage. The receptacles 223 can be removably attached to a body 301. The body 301 can be configured to receive one, two, or more receptacles 223. In at least one example, as illustrated in FIG. 2A, the body 301 can be substantially circular in shape. In other examples, the body 301 can be rectangular, triangular, polygonal, or any other suitable shape.

In at least one example, the system 201 can include a separation component 260 which can sort the embryos 207 into a desired receptacle 223 based on the properties of the embryo 207. For example, the separation component 260 may separate viable embryos into one receptacle 230 and unviable embryos into another receptacle 230. As such, an operator can pass a plurality of embryos through the system 201 and have the embryos 207 be sorted and organized by the desired properties. In at least one example, the separation component 260 can be in communication with the processor 2200 such that the processor 2200 automatically instructs the separation component 260 to direct the embryos into the desired receptacle(s) 223. In some examples, an operator may trigger the separation component 260 as the results of the testing become known and the embryos 207 have not yet reached the bottom of the measurement chamber 203.

In at least one example, the separation component 260 can be positioned within the annulus 202 of the measurement chamber 203 proximate to the second end 229. In other examples, the separation component 260 can be disposed outside of the measurement chamber 203 proximate to the second end 229 and positioned between the second end 229 of the measurement chamber 203 and the receptacle(s) 223. The separation component 260 is in communication with the annulus 202 such that the embryos 207 pass through the separation component 260 to the receptacle(s) 223.

In at least one example, the separation component 260 can include a valve which rotates to direct the embryos into the desired receptacle(s) 223. In other examples, the separation component may include a flow cytometer which circulates the culture medium 225 to separate and direct embryos 207 into piles and/or desired receptacle(s) 223.

In at least one example, as illustrated in FIG. 2A, the system 201 can be situated in a controlled environment 270. The controlled environment 270 can be, for example, a housing configured to entirely contain measurement chamber 203. As in the illustrated example, controlled environment 270 can include a control system 240 which can include one or more components configured to control the environment. For example, the components can include temperature regulation component 242 and a UV control component 244. Additionally, the controlled environment 270 can provide protection from contamination.

In at least one example, the system 201 can further include a heating pad/plate 252 configured to provide a warming to storage platform 205. In addition, a catch plate 250 can be incorporated to ensure that any loss of embryos 207 from the measurement chamber 203 and/or the receptacles 223 is retained within an area.

FIG. 2B is a block diagram of an exemplary processing system 221. Processing system 221 is configured to perform processing of data and communicate with the sensors 212, for example as illustrated in FIG. 2A. In operation, processing system 221 communicates with one or more of the above-discussed components and may also be configured to communication with remote devices/systems.

As shown, processing system 221 includes hardware and software components such as network interfaces 2100, at least one processor 2200, sensors 2600 and a memory 2400 interconnected by a system bus 2500. Network interface(s) 2100 can include mechanical, electrical, and signaling circuitry for communicating data signals over communication links, which may include wired or wireless communication links. Network interfaces 2100 are configured to transmit and/or receive data signals using a variety of different communication protocols, as will be understood by those skilled in the art.

Processor 2200 represents a digital signal processor (e.g., a microprocessor, a microcontroller, or a fixed-logic processor, etc.) configured to execute instructions or logic to perform tasks in a wellbore environment. Processor 2200 may include a general purpose processor, special-purpose processor (where software instructions are incorporated into the processor), a state machine, application specific integrated circuit (ASIC), a programmable gate array (PGA) including a field PGA, an individual component, a distributed group of processors, and the like. Processor 2200 typically operates in conjunction with shared or dedicated hardware, including but not limited to, hardware capable of executing software and hardware. For example, processor 2200 may include elements or logic adapted to execute software programs and manipulate data structures 2450, which may reside in memory 2400.

Sensors 2600, which may include sensors 212 as disclosed herein, typically operate in conjunction with processor 2200 to perform measurements, and can include special-purpose processors, detectors, transmitters, receivers, and the like. In this fashion, sensors 2600 may include hardware/software for generating, transmitting, receiving, detection, logging, and/or sampling magnetic fields, seismic activity, and/or acoustic waves, or other parameters.

Memory 2400 comprises a plurality of storage locations that are addressable by processor 2200 for storing software programs and data structures 2450 associated with the embodiments described herein. An operating system 2420, portions of which may be typically resident in memory 2400 and executed by processor 2200, functionally organizes the device by, inter alia, invoking operations in support of software processes and/or services 2440 executing on processing system 221. These software processes and/or services 2440 may perform processing of data and communication with processing system 221, as described herein. Note that while process/service 2440 is shown in centralized memory 2400, some examples provide for these processes/services to be operated in a distributed computing network.

It will be apparent to those skilled in the art that other processor and memory types, including various computer-readable media, may be used to store and execute program instructions pertaining to the fluidic channel evaluation techniques described herein. Also, while the description illustrates various processes, it is expressly contemplated that various processes may be embodied as modules having portions of the process/service 2440 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 2200 or computer readable medium encoded with instructions for execution by processor 2200 that, when executed by the processor, are operable to cause the processor to perform the functions described herein.

In at least one example, the processor 2200 can apply machine learning, such as a neural network or sequential logistic regression and the like, to determine relationships between the data signals from the sensor(s) 212 and properties of the embryos 207. For example, a deep neural network may be trained in advance to capture the complex relationship between the descent rate and/or size of the embryo 207 and the viability and/or sex of the embryo 207. Additionally or alternatively, in at least one example, the processor 2200 can apply image processing. With image processing, the processor 2200 can process images that the sensors 212 may provide to assess the embryos 207 descending through the measurement chamber 203. For example, the sensors 212 may include a camera which provides images of at least a portion of the measurement chamber 203. The camera transmits the images to the processor 2200 which performs image processing to locate the embryos and track the descent of the embryos. Also, in some examples, the processor 2200, with image processing, can assess the embryos in regards to other characteristics such as diameter or shape. In at least one example, with image processing, a user is not needed to assess the descending embryos. Additionally, if a plurality of embryos is disposed within the measurement chamber simultaneously, the sensors 212 and the processor 2200 can assess each embryo 207. As such, the determination of properties of the embryos 207 can be more accurate.

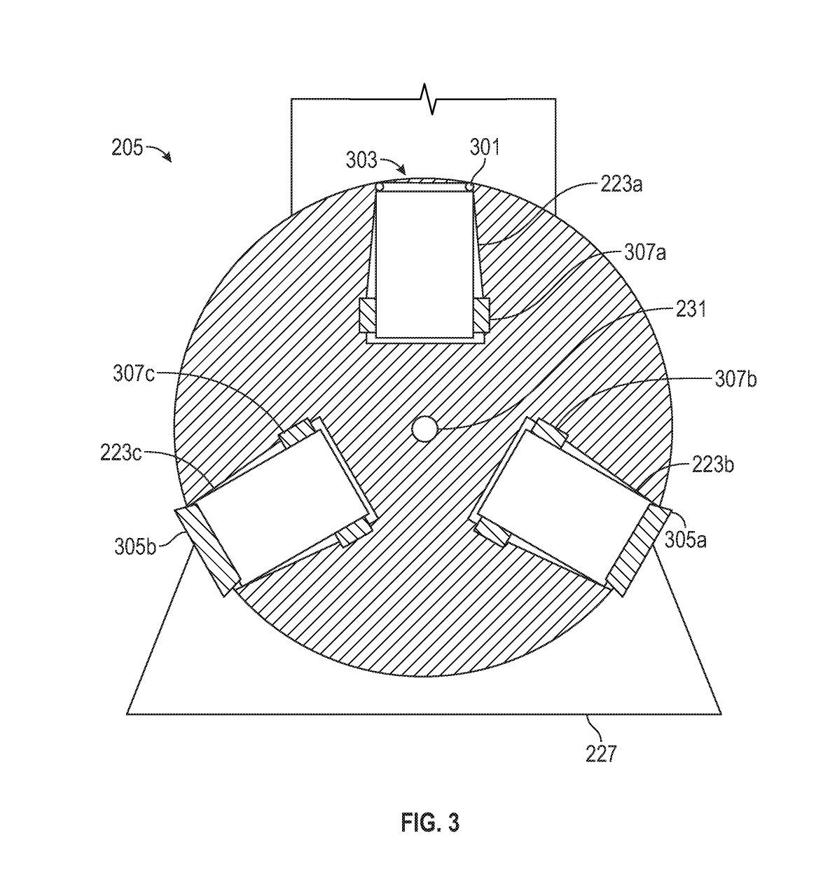

FIG. 3 illustrates an exemplary storage platform 205. As illustrated in FIG. 3, each receptacle 223 can have a mating seal 301 at an opening 303. In at least one example, the receptacles 223 can include a lid 305 removably attached over the opening 303. The lid 305 can be coupled to the receptacle 223 by friction force. In at least one example, the lid 305 can also be coupled to the receptacle 223 by a hinge. Holders 307a, 307b, 307c receive the corresponding receptacles 223a, 223b, 223c. The receptacles 223 are in removable communication with the storage platform 205 via the holders 307. The number of holders 307 can be one, two, or more holders as desired. While the holders 307 are depicted as a press fit rubber pad any device for holding the receptacles 223 in the storage platform 205 can be implemented. The storage platform 205, as illustrated in FIG. 3, can be rotationally attached to a stand 227 via an axel 231. As such, the storage platform 205 can rotate about the axel 231 to align the desired receptacle 223 with the measurement chamber 203. The receptacles 223 can be filled with culture medium 225 such that the embryos 207 can remain viable while contained within the receptacles 223.

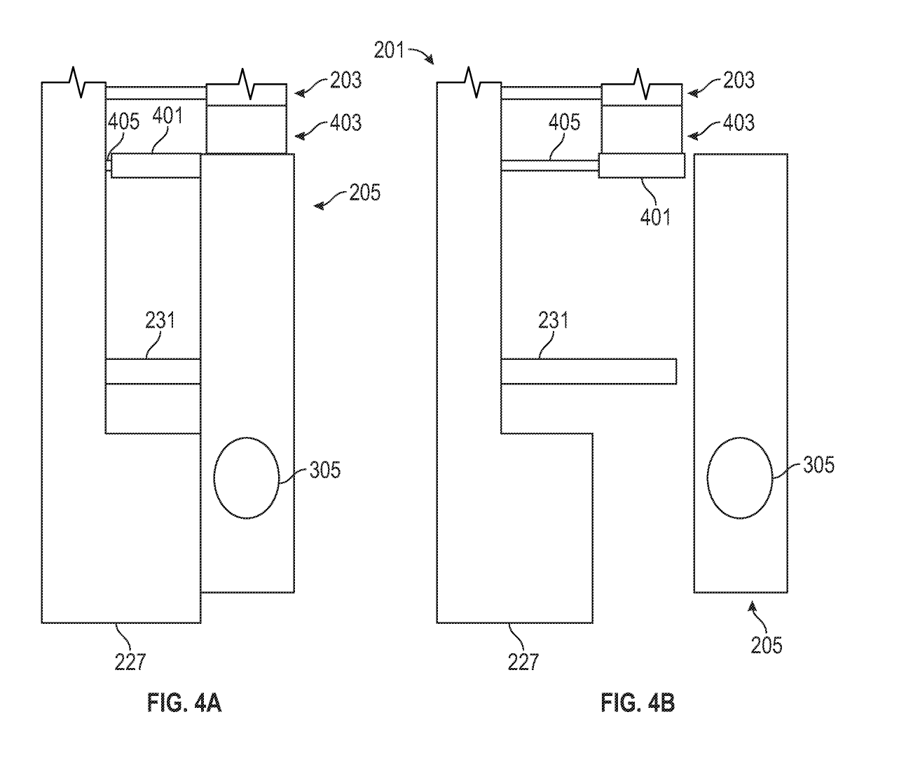

FIGS. 4A and 4B illustrate an exemplary system which can close the second end 229 of the measurement chamber 203 such that culture medium 225 does not spill when the receptacles 223 are moved. As illustrated in FIGS. 4A and 4B, the system 201 can include stand 227 supporting measurement chamber 203 and storage platform 205. A bottom door 401 can be retractably coupled to the support 227 via a spring-loaded extension arm 405 at the bottom end 403 of the measurement chamber 203. As the storage platform 205 and/or the receptacle 223 is removed from being aligned with the measurement chamber 203, the bottom door 401 follows behind, closing the bottom end 403 of the measurement chamber 203 and preventing the loss of culture medium 225. Receptacles 223 can accept indicia that associate the contained embryo 207 with the results of the test performed. Other suitable methods or systems to prevent loss of culture medium 225 from the measurement chamber 203 can also be implemented without deviating from the scope of the disclosure.

Through the use of the system 201, embryos 207 can be identified and/or separated so that the embryos 207 can be selected based on the results of the test. Additionally, the body 301, for example by being a substantially circular shape, can act as a seal while the measurement chamber 203 prevents loss of culture medium 225 while changing receptacles 223 between tests.

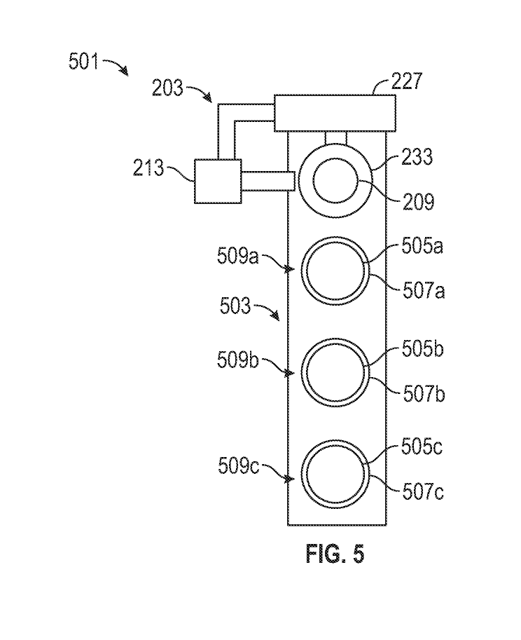

FIG. 5 illustrates another example of the system 201, system 501. Any features of system 201 and system 501 may be interchangeable between the systems 201, 501. System 501 includes a measurement chamber 203 in fluid communication with a storage platform 503. The storage platform 503 includes a plurality of receptacles 505 rigidly attached to each other. The receptacles 505 are arranged along the same plane. Each receptacle 505 can have a mating seal 507 rigidly attached to an opening 509. In at least one example, the measurement chamber 203 can be moved to transition between receptacles 505. In other examples, the storage platform 503 can be moved to transition between receptacles 505. The transition between receptacles 505 could be manual or automatic. In yet other examples, the measurement chamber 203 can be in fluid communication with a plurality of receptacles 505 without the need to move either the measurement chamber 203 or the storage platform 503.

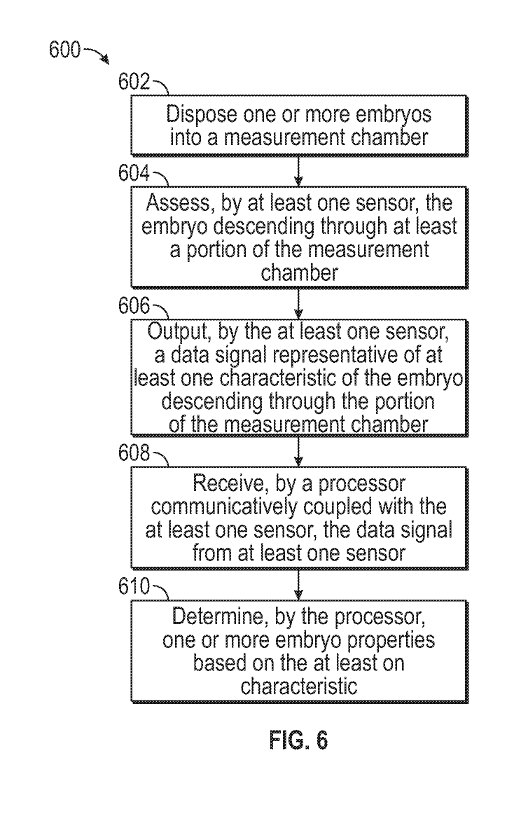

Referring to FIG. 6, a flowchart is presented in accordance with an example embodiment. The method 600 is provided by way of example, as there are a variety of ways to carry out the method. The method 600 described below can be carried out using the configurations illustrated in FIG. 1-5, for example, and various elements of these figures are referenced in explaining example method 600. Each block shown in FIG. 6 represents one or more processes, methods or subroutines, carried out in the example method 600. Furthermore, the illustrated order of blocks is illustrative only and the order of the blocks can change according to the present disclosure. Additional blocks may be added or fewer blocks may be utilized, without departing from this disclosure. The example method 600 can begin at block 602.

At block 602, one or more embryos are disposed into a measurement chamber of a system. The measurement chamber can include a culture medium which fills up at least a portion of the measurement chamber. The culture medium provides an environment such that the embryos can grow and/or maintain viability. The embryos, after being disposed into the measurement chamber, descend towards a bottom end of the measurement chamber.

At block 604, at least one sensor assesses the embryos descending through at least a portion of the measurement chamber. The sensor can be, for example, a camera which visibly senses the embryos. In other examples, the sensor may be able to sense the embryos without direct visibility. In at least one example, the sensor can locate the embryos and track the movement of the embryos.

At block 606, the at least one sensor outputs a data signal representative of at least one characteristic of the embryo descending through the portion of the measurement chamber. The characteristic that the sensor assesses and outputs can include, for example, a descent rate of the embryos. Additionally, in some examples, the characteristic can include embryo diameter. Other suitable characteristics which can be assessed and/or measured which provides information about the embryo can be measured by the sensor(s).

At block 608, a processor, communicatively coupled with the sensor, receives the data signal from the sensor. In at least one example, the processor is directly coupled with the sensor. In some examples, the processor can be separate from the system. At block 610, the processor determines one or more embryo properties based on the at least one characteristic of the embryo descending through the measurement chamber. For example, the properties can include one or more of: embryo viability, embryo sex, embryo development potential, embryo biochemical composition, oocyte competency, embryo survival of cryopreservation, aneuploidy, or trisomy.

The embryos, after passing through the measurement chamber, can be stored. In at least one example, the embryos can be stored within the measurement chamber. In other examples, the embryos can be received and stored within one or more receptacles. Each of the receptacles can be sealed after the receptacle has received one or more embryos as desired. The position of the storage platform can be moved with respect to the measurement chamber to receive additional embryos in the receptacles as the embryos exit the measurement chamber.

In some examples, the measurement chamber may be in communication with a plurality of receptacles without the need to move the storage platform, and the embryos are sorted into the desired receptacles by a separation component based on the one or more embryo properties. For example, the separation component can include a valve which rotates to direct the one or more embryos into the desired receptacle. In other examples, the separation component can include a flow cytometer.

After the embryos with the desired properties have been selected and/or sorted, the embryos can be implanted into a female for offspring. The embryos can also be frozen and saved for later use.

Numerous examples are provided herein to enhance understanding of the present disclosure. A specific set of statements are provided as follows.

Statement 1: A system is disclosed to evaluate an embryo, the system comprising: a measurement chamber having a bottom end, the measurement chamber being configured to receive an embryo such that the embryo descends towards the bottom end; a culture medium disposed within the measurement chamber; at least one sensor configured to assess the embryo descending towards the bottom end and output a data signal representative of at least one characteristic of the embryo descending through at least a portion of the measurement chamber; a processor communicatively coupled with the at least one sensor; and a memory configured to store instructions executable by the processor, the instructions, when executed, are operable to:

receive the data signal from the at least one sensor; and determine one or more embryo properties based on the at least one characteristic.

Statement 2: A system is disclosed according to Statement 1, wherein the at least one characteristic includes a descent rate of the embryo.

Statement 3: A system is disclosed according to Statements 1 or 2, wherein the one or more embryo properties includes embryo viability.

Statement 4: A system is disclosed according to any of preceding Statements 1-3, wherein the one or more embryo properties includes embryo sex.

Statement 5: A system is disclosed according to any of preceding Statements 1-4, wherein the one or more embryo properties includes one or more of embryo development potential, embryo biochemical composition, oocyte competency, embryo survival of cryopreservation, aneuploidy, or trisomy.

Statement 6: A system is disclosed according to any of preceding Statements 1-5, wherein the at least one characteristic includes a diameter of the embryo.

Statement 7: A system is disclosed according to any of preceding Statements 1-6, wherein the at least one sensor is configured to located the embryo and track the descent of the embryo.

Statement 8: A system is disclosed according to any of preceding Statements 1-7, wherein the at least one sensor includes a camera.

Statement 9: A system is disclosed according to any of preceding Statements 1-8, further comprising: at least one receptacle configured to store the embryo.

Statement 10: A system is disclosed according to Statement 9, further comprising: a separation component which sorts the embryo into a desired receptacle of the at least one receptacle based on the one or more embryo properties.

Statement 11: A system is disclosed according to Statement 10, wherein the separation component includes a valve which rotates to direct the embryo into the desired receptacle.

Statement 12: A system is disclosed according to Statements 10 or 11, wherein the separation component includes a flow cytometer.

Statement 13: A method is disclosed comprising: disposing an embryo into a measurement chamber which includes a culture medium, the embryo descending towards a bottom end of the measurement chamber; assessing, by at least one sensor, the embryo descending through at least a portion of the measurement chamber; outputting, by the at least one sensor, a data signal representative of at least one characteristic of the embryo descending through the portion of the measurement chamber; receiving, by a processor communicatively coupled with the at least one sensor, the data signal from the at least one sensor; and determining, by the processor, one or more embryo properties based on the at least one characteristic.

Statement 14: A method is disclosed according to Statement 13, wherein the at least one characteristic includes a descent rate of the embryo.

Statement 15: A method is disclosed according to Statements 13 or 14, wherein the one or more embryo properties includes one or more of embryo viability, embryo sex, embryo development potential, embryo biochemical composition, oocyte competency, embryo survival of cryopreservation, aneuploidy, or trisomy.

Statement 16: A method is disclosed according to any of preceding Statements 13-15, wherein the at least one characteristic includes a diameter of the embryo.

Statement 17: A method is disclosed according to any of preceding Statements 13-16, wherein assessing the embryo by the at least one sensor further comprises: locating, by the at least one sensor, the embryo; and tracking, by the at least one sensor, the descent of the embryo.

Statement 18: A method is disclosed according to any of preceding Statements 13-17, wherein the at least one sensor includes a camera.

Statement 19: A method is disclosed according to any of preceding Statements 13-18, further comprising: sorting, by a separation component, the embryo into a desired receptacle based on the one or more embryo properties.

Statement 20: A method is disclosed according to Statement 19: wherein the separation component includes a valve which rotates to direct the embryo into the desired receptacle.

Statement 21: A method is disclosed according to Statements 19 or 20, wherein the separation component includes a flow cytometer.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

1. A system to evaluate an embryo, the system comprising:
a measurement chamber having a bottom end, the measurement chamber being configured to receive an embryo such that the embryo descends towards the bottom end;
a culture medium disposed within the measurement chamber;
at least one sensor configured to assess the embryo descending towards the bottom end and output a data signal representative of at least one characteristic of the embryo descending through at least a portion of the measurement chamber;
a processor communicatively coupled with the at least one sensor; and
a memory configured to store instructions executable by the processor, the instructions, when executed, are operable to:
receive the data signal from the at least one sensor; and
determine one or more embryo properties based on the at least one characteristic.

2. The system of claim 1, wherein the at least one characteristic includes a descent rate of the embryo.

3. The system of claim 1, wherein the one or more embryo properties includes embryo viability.

4. The system of claim 1, wherein the one or more embryo properties includes embryo sex.

5. The system of claim 1, wherein the one or more embryo properties includes one or more of embryo development potential, embryo biochemical composition, oocyte competency, embryo survival of cryopreservation, aneuploidy, or trisomy.

6. The system of claim 1, wherein the at least one characteristic includes a diameter of the embryo.

7. The system of claim 1, wherein the at least one sensor is configured to locate the embryo and track the descent of the embryo.

8. The system of claim 1, wherein the at least one sensor includes a camera.

9. The system of claim 1, further comprising: at least one receptacle configured to store the embryo.

10. The system of claim 9, further comprising: a separation component which sorts the embryo into a desired receptacle of the at least one receptacle based on the one or more embryo properties.

11. The system of claim 10, wherein the separation component includes a valve which rotates to direct the embryo into the desired receptacle.

12. The system of claim 10, wherein the separation component includes a flow cytometer.

13. A method comprising:
disposing an embryo into a measurement chamber which includes a culture medium, the embryo descending towards a bottom end of the measurement chamber;
assessing, by at least one sensor, the embryo descending through at least a portion of the measurement chamber;
outputting, by the at least one sensor, a data signal representative of at least one characteristic of the embryo descending through the portion of the measurement chamber;
receiving, by a processor communicatively coupled with the at least one sensor, the data signal from the at least one sensor; and
determining, by the processor, one or more embryo properties based on the at least one characteristic.

14. The method of claim 13, wherein the at least one characteristic includes a descent rate of the embryo.

15. The method of claim 13, wherein the one or more embryo properties includes one or more of embryo viability, embryo sex, embryo development potential, embryo biochemical composition, oocyte competency, embryo survival of cryopreservation, aneuploidy, or trisomy.

16. The method of claim 13, wherein the at least one characteristic includes a diameter of the embryo.

17. The method of claim 13, wherein assessing the embryo by the at least one sensor further comprises:
locating, by the at least one sensor, the embryo; and
tracking, by the at least one sensor, the descent of the embryo.

18. The method of claim 13, wherein the at least one sensor includes a camera.

19. The method of claim 13, further comprising:
sorting, by a separation component, the embryo into a desired receptacle based on the one or more embryo properties.

20. The method of claim 19, wherein the separation component includes a valve which rotates to direct the embryo into the desired receptacle.

21. The method of claim 19, wherein the separation component includes a flow cytometer.
